(19) United States
(12) Patent Application Publication
(10) Pub. No.: US 2022/0162306 A1
(43) Pub. Date: May 26, 2022

(54) METHODS AND COMPOSITIONS FOR TREATMENT OF LUPUS

(71) Applicant: Provention Bio, Inc., Red Bank, NJ (US)

(72) Inventors: Francisco Leon, Red Bank, NJ (US); Paul Dunford, Red Bank, NJ (US); Paul Moore, Rockville, MD (US)

(73) Assignee: MacroGenics, Inc., Rockville, MD (US)

(21) Appl. No.: 17/453,151

(22) Filed: Nov. 1, 2021

Related U.S. Application Data
(60) Provisional application No. 63/108,406, filed on Nov. 1, 2020.

(51) Int. Cl. C07K 16/28; C07K 16/46

(52) U.S. Cl. CPC C07K 16/283; A61K 2039/505; C07K 16/468

(57) ABSTRACT

Disclosed herein, in one aspect, is a method of treating B cell driven autoimmune and allergic diseases, such as lupus, comprising administering to a patient in need thereof an effective amount of B cell inhibitor that is non-depletional. Related compositions are also provided

### CROSS REFERENCE TO RELATED APPLICATIONS

This application claims priority to and the benefit of U.S. Provisional Patent Application No. 63/108,406 filed Nov. 1, 2020, the entire disclosure of which is incorporated herein by reference.

### SEQUENCE LISTING

The ASCII text file submitted herewith via EFS-Web, entitled “011001seq.txt” created on Nov. 1, 2021, having a size of 13,219 bytes, is incorporated herein by reference in its entirety.

### FIELD

The present disclosure generally relates to compositions and methods for the treatment of lupus, and more particularly to do so by means of B cell inhibitors.

### BACKGROUND

Systemic Lupus Erythematosus (SLE) is a chronic, multi-organ autoimmune disease with significant impact on survival, disability, and quality of life. The disease primarily affects women of childbearing age, but all age groups may be affected. According to the Lupus Foundation of America (LFA), more than 16,000 new cases of lupus are reported annually across the United States with a prevalence of at least 1.5 million in the US and 5 million worldwide (LFA 2014).

SLE is highly variable both in clinical presentation and course of the disease (Bartels 2014). Comorbidities of the disease and side effects of treatment increase the risk of morbidity and mortality in patients with SLE (Bertsias 2008). Despite major advances in understanding the pathogenesis and clinical course of lupus and improvements in overall survival, the general prognosis for lupus patients remains poor with high direct and indirect costs of the disease.

SLE is characterized by the emergence and persistence of pathogenic subsets of B cells and autoantibodies against multiple autoantigens, leading to unpredictable flares of inflammation in the skin, joints, and other tissues (Bartels 2014; Cancro 2009). There have been few approved treatments developed specifically for lupus, and patients must frequently rely on older, untested immune modulators with significant safety and tolerability issues to control their disease.

As such, a need exists for methods and compositions for the treatment of lupus.

### SUMMARY

Disclosed herein, in one aspect, is a method of treating B cell driven autoimmune and/or allergic diseases, such as lupus, comprising administering to a subject in need thereof a B cell inhibitor that is non-depletional. In some embodiments, the subject has reduced CD32B signaling compared to a healthy subject, wherein the B cell inhibitor is capable of agonizing CD32B despite the reduced CD32B signaling.

Also disclosed herein is a composition for use as a medicament for the treatment of B cell driven autoimmune and/or allergic diseases, such as lupus, comprising a B cell inhibitor that is non-depletional. In some embodiments, the composition can be used to treat a subject that has reduced CD32B signaling compared to a healthy subject, wherein the B cell inhibitor is capable of agonizing CD32B despite the reduced CD32B signaling.

Further disclosed herein is use of a composition for the manufacture of a medicament for the treatment of B cell driven autoimmune and/or allergic diseases, such as lupus, wherein the composition includes a B cell inhibitor that is non-depletional. In some embodiments, the composition can be used to treat a subject that has reduced CD32B signaling compared to a healthy subject, wherein the B cell inhibitor is capable of agonizing CD32B despite the reduced CD32B signaling.

In some embodiments, the B cell inhibitor is a CD32B×CD79B bi-specific antibody capable of immunospecifically binding an epitope of CD32B and an epitope of CD79B. In some embodiments, the CD32B×CD79B bi-specific antibody comprises:

(A) a VL_{CD32B} domain that comprises the amino acid sequence of SEQ ID NO: 1;
(B) a VH_{CD32B} domain that comprises the amino acid sequence of SEQ ID NO: 2;
(C) a VL_{CD79B} domain that comprises the amino acid sequence of SEQ ID NO: 3; and
(D) a VH_{CD79B} domain that comprises the amino acid sequence of SEQ ID NO: 4.

In some embodiments, the CD32B×CD79B bi-specific antibody is an Fc diabody comprising:

(A) a first polypeptide chain that comprises the amino acid sequence of SEQ ID NO: 5;
(B) a second polypeptide chain that comprises the amino acid sequence of SEQ ID NO: 6; and
(C) a third polypeptide chain that comprises the amino acid sequence of SEQ ID NO: 7.

In some embodiments, the Fc diabody can be administered at a dose of between about 5 mg/kg and about 40 mg/kg, and at a dosage regimen of between one dose per 2 to 8 weeks, for a total of 2 to 20 doses. In some embodiments, the Fc diabody can be administered at a dose of about 5 to 20 mg/kg, and at a dosage regimen of one dose every 2 to 6 weeks for a total of 5 to 10 doses. In some embodiments, the Fc diabody can be administered at a dose of about 10 mg/kg and at a dosage regimen of one dose per 2 to 4 weeks for a total of 6 to 8 doses. In some embodiments, the Fc diabody can be administered at a dose of about 10 mg/kg and at a dosage regimen of one dose every 4 weeks for a total of 6 doses.

In some embodiments, the Fc diabody can be administered via an intravenous infusion. In some embodiments, the Fc diabody can be administered over a period of about 1-10 hours, or about 2-4 hours, or about 2 hours.

In some embodiments, the Fc diabody can be administered 10 mg/kg IV or SC equivalent once every 4 weeks, indefinitely (e.g., chronic therapy).

In some embodiments, the disease can be selected from Systemic Lupus Erythematosus (SLE), Multiple Sclerosis (MS), Rheumatoid Arthritis (RA), Psoriasis, Dermatomyositis/Polymyositis, Sjogren's Syndrome (SS), Primary Vasculitis (e.g. Polymyalgia rheumatica/Giant cell arteritis/Behçets), Graft vs. Host Disease (GVHD), Myasthenia Gravis, Pemphigus, Neuromyelitis Optica, Anti-NMDA receptor encephalitis, Guillain-Barré syndrome, Chronic inflammatory demyelinating polyneuropathy (CIDP), Grave's opthalmopathy, IgG4 Related Disease (IgG4-RD), Idiopathic thrombocytopenic purpura (ITP), Inflammatory Bowel Disease (IBD), and Crohn's Disease. In some embodiments, the disease is Systemic Lupus Erythematosus.

Also provided herein are pharmaceutical compositions comprising the B cell inhibitors disclosed herein, provided (e.g., packaged) at therapeutically effective unit doses. Instructions for dosage regimens as disclosed herein can also be provided.

### BRIEF DESCRIPTION OF THE DRAWINGS

The patent or application file contains at least one drawing executed in color. Copies of this patent or patent application publication with color drawing(s) can be provided by the Office upon request and payment of the necessary fee.

FIG. 1: Dose proportional PK upon repeat dosing.

FIGS. 2A-2B: No sustained reduction in % (FIG. 2A) or total (FIG. 2B) B cells after repeat dosing of PRV-3279.

FIGS. 3A-3B: Dose dependent proportion of B cells bound (FIG. 3A) and intensity of binding (FIG. 3B).

FIGS. 4A-4B: Durable, dose dependent reduction in IgM (FIG. 4A) and reduction in IgE (FIG. 4B).

FIG. 5: Time and dose dependent inhibition of ADA development.

FIG. 6: Inhibition of B cells from normal subjects (panel A) or SLE patients (panel B).

FIG. 7: A schematic of a phase 2a study design.

### DETAILED DESCRIPTION

Disclosed herein, in one aspect, is a method of B cell driven autoimmune and/or allergic diseases, such as lupus, comprising administering to a subject in need thereof a non-depleting functional inhibitor of B cells. In some embodiments, the B cell inhibitor is a CD32B×CD79B bi-specific antibody such as those disclosed in U.S. Publication No. 2016/0194396, WIPO Publication Nos. WO 2015/021089 and WO2017/214096, each incorporated by reference in its entirety.

Polymorphisms in CD32B in humans are associated with an increased prevalence of SLE (Chen et al., Association of a transmembrane polymorphism of Fcgamma receptor IIb (FCGR2B) with systemic lupus erythematosus in Taiwanese patients. Arthritis Rheum. 2006; 54(12):3908-3917), and there is evidence of reduced CD32B signaling in at least a subset of lupus patients (Floto et al., Loss of function of a lupus-associated FcgammaRIIb polymorphism through exclusion from lipid rafts. Nat Med. 2005; 11(10):1056-1058). Surprisingly, as disclosed herein, PRV-3279 is shown to be able to suppress the function of B cells from lupus patients to the same extent as those from healthy controls. Thus, PRV-3279 is able to agonize or activate the CD32B pathway in lupus despite the reduced CD32B signaling.

### Definitions

For convenience, certain terms employed in the specification, examples, and appended claims are collected here. Unless defined otherwise, all technical and scientific terms used herein have the same meaning as commonly understood by one of ordinary skill in the art to which this disclosure belongs.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the method/device being employed to determine the value, or the variation that exists among the study subjects. Typically the term is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% variability depending on the situation.

The term “substantially” means more than 50%, preferably more than 80%, and most preferably more than 90% or 95%.

The use of the term “or” in the claims is used to mean “and/or” unless explicitly indicated to refer only to alternatives or the alternatives are mutually exclusive, although the disclosure supports a definition that refers to only alternatives and “and/or.”

As used in this specification and claim(s), the terms “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited, elements or method steps. It is contemplated that any embodiment discussed in this specification can be implemented with respect to any method, system, host cells, expression vectors, and/or composition of the invention. Furthermore, compositions, systems, host cells, and/or vectors of the invention can be used to achieve methods and proteins of the invention.

As used herein the term “consisting essentially of” refers to those elements required for a given embodiment. The term permits the presence of additional elements that do not materially affect the basic and novel or functional characteristic(s) of that embodiment of the disclosure.

The term “consisting of” refers to compositions, methods, and respective components thereof as described herein, which are exclusive of any element not recited in that description of the embodiment.

The use of the term “for example” and its corresponding abbreviation “e.g.” (whether italicized or not) means that the specific terms recited are representative examples and embodiments of the invention that are not intended to be limited to the specific examples referenced or cited unless explicitly stated otherwise.

“Antibody” or “antibody molecule” as used herein refers to a protein, e.g., an immunoglobulin chain or fragment thereof, comprising at least one immunoglobulin variable domain sequence. An antibody molecule encompasses antibodies (e.g., full-length antibodies) and antibody fragments. In an embodiment, an antibody molecule comprises an antigen binding or functional fragment of a full length antibody, or a full length immunoglobulin chain. For example, a full-length antibody is an immunoglobulin (Ig) molecule (e.g., IgG) that is naturally occurring or formed by normal immunoglobulin gene fragment recombinatorial processes). In embodiments, an antibody molecule refers to an immunologically active, antigen-binding portion of an immunoglobulin molecule, such as an antibody fragment. An antibody fragment, e.g., functional fragment, is a portion of an antibody, e.g., Fab, Fab′, F(ab′)₂, F(ab)₂, variable fragment (Fv), domain antibody (dAb), or single chain variable fragment (scFv). A functional antibody fragment binds to the same antigen as that recognized by the intact (e.g., full-length) antibody. The terms “antibody fragment” or “functional fragment” also include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). In some embodiments, an antibody fragment does not include portions of antibodies without antigen binding activity, such as Fc fragments or single amino acid residues. Exemplary antibody molecules include full length antibodies and antibody fragments, e.g., dAb (domain antibody), single chain, Fab, Fab′, and F(ab′)₂ fragments, and single chain variable fragments (scFvs). The terms “Fab” and “Fab fragment” are used interchangeably and refer to a region that includes one constant and one variable domain from each heavy and light chain of the antibody, i.e., V_{L}, C_{L}, V_{H}, and C_{H}1.

Throughout the present specification, the numbering of the residues in the constant region of an IgG Heavy Chain is that of the EU index as in Kabat et al., Sequences of Proteins of Immunological Interest, 5^{th} Ed. Public Health Service, NH1, MD (1991) (“Kabat”), expressly incorporated herein by references. The term “EU index as in Kabat” refers to the numbering of the human IgG1 EU antibody. Amino acids from the Variable Domains of the mature heavy and Light Chains of immunoglobulins are designated by the position of an amino acid in the chain. Kabat described numerous amino acid sequences for antibodies, identified an amino acid consensus sequence for each subgroup, and assigned a residue number to each amino acid, and the CDRs are identified as defined by Kabat (it can be understood that CDR_{H}1 as defined by Chothia, C. & Lesk, A. M. ((1987) “Canonical structures for the hypervariable regions of immunoglobulins,”. J. Mol. Biol. 196:901-917) begins five residues earlier). Kabat's numbering scheme is extendible to antibodies not included in his compendium by aligning the antibody in question with one of the consensus sequences in Kabat by reference to conserved amino acids. This method for assigning residue numbers has become standard in the field and readily identifies amino acids at equivalent positions in different antibodies, including chimeric or humanized variants. For example, an amino acid at position 50 of a human antibody Light Chain occupies the equivalent position to an amino acid at position 50 of a mouse antibody Light Chain.

In embodiments, an antibody molecule is monospecific, e.g., it comprises binding specificity for a single epitope. In some embodiments, an antibody molecule is multispecific, e.g., it comprises a plurality of immunoglobulin variable domain sequences, where a first immunoglobulin variable domain sequence has binding specificity for a first epitope and a second immunoglobulin variable domain sequence has binding specificity for a second epitope. In some embodiments, an antibody molecule is a bispecific antibody molecule.

The terms “bispecific antibody molecule,” “diabody” and DART® protein are used interchangeably herein and refer to an antibody molecule that has specificity for more than one (e.g., two, three, four, or more) epitope and/or antigen. In some embodiments, the antibody can be diabodies or scaffolds capable of antigen binding, such as those disclosed in U.S. Publication No. 2016/0194396, WIPO Publication Nos. WO 2015/021089 and WO2017/214096, each incorporated by reference in its entirety. In some embodiments, the antibody can be CD32B×CD79B bispecific diabodies (i.e., “CD32B×CD79B diabodies,” and such diabodies that additionally comprise an Fc domain (i.e., “CD32B×CD79B Fc diabodies”). In one embodiment, the antibody can be a humanized CD32B×CD79B DART® protein, produced in Chinese hamster ovary cells with a molecular weight of 111.5 kDa.

“Antigen” (Ag) as used herein refers to a macromolecule, including all proteins or peptides. In some embodiments, an antigen is a molecule that can provoke an immune response, e.g., involving activation of certain immune cells and/or antibody generation. Antigens are not only involved in antibody generation. T cell receptors also recognized antigens (albeit antigens whose peptides or peptide fragments are complexed with an MHC molecule). Any macromolecule, including almost all proteins or peptides, can be an antigen. Antigens can also be derived from genomic recombinant or DNA. For example, any DNA comprising a nucleotide sequence or a partial nucleotide sequence that encodes a protein capable of eliciting an immune response encodes an “antigen.” In embodiments, an antigen does not need to be encoded solely by a full length nucleotide sequence of a gene, nor does an antigen need to be encoded by a gene at all. In embodiments, an antigen can be synthesized or can be derived from a biological sample, e.g., a tissue sample, a tumor sample, a cell, or a fluid with other biological components. As used, herein a “tumor antigen” or interchangeably, a “cancer antigen” includes any molecule present on, or associated with, a cancer, e.g., a cancer cell or a tumor microenvironment that can provoke an immune response. As used, herein an “immune cell antigen” includes any molecule present on, or associated with, an immune cell that can provoke an immune response.

The “antigen-binding site” or “antigen-binding fragment” or “antigen-binding portion” (used interchangeably herein) of an antibody molecule refers to the part of an antibody molecule, e.g., an immunoglobulin (Ig) molecule such as IgG, that participates in antigen binding. In some embodiments, the antigen-binding site is formed by amino acid residues of the variable (V) regions of the heavy (H) and light (L) chains. Three highly divergent stretches within the variable regions of the heavy and light chains, referred to as hypervariable regions, are disposed between more conserved flanking stretches called “framework regions” (FRs). FRs are amino acid sequences that are naturally found between, and adjacent to, hypervariable regions in immunoglobulins. In embodiments, in an antibody molecule, the three hypervariable regions of a light chain and the three hypervariable regions of a heavy chain are disposed relative to each other in three dimensional space to form an antigen-binding surface, which is complementary to the three-dimensional surface of a bound antigen. The three hypervariable regions of each of the heavy and light chains are referred to as “complementarity-determining regions,” or “CDRs.” The framework region and CDRs have been defined and described, e.g., in Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917. Each variable chain (e.g., variable heavy chain and variable light chain) is typically made up of three CDRs and four FRs, arranged from amino-terminus to carboxy-terminus in the amino acid order: FR1, CDR1, FR2, CDR2, FR3, CDR3, and FR4. Variable light chain (VL) CDRs are generally defined to include residues at positions 27-32 (CDR1), 50-56 (CDR2), and 91-97 (CDR3). Variable heavy chain (VH) CDRs are generally defined to include residues at positions 27-33 (CDR1), 52-56 (CDR2), and 95-102 (CDR3). One of ordinary skill in the art would understand that the loops can be of different length across antibodies and the numbering systems such as the Kabat or Chotia control so that the frameworks have consistent numbering across antibodies.

In some embodiments, the antigen-binding fragment of an antibody (e.g., when included as part of a fusion molecule) can lack or be free of a full Fc domain. In certain embodiments, an antibody-binding fragment does not include a full IgG or a full Fc but may include one or more constant regions (or fragments thereof) from the light and/or heavy chains. In some embodiments, the antigen-binding fragment can be completely free of any Fc domain. In some embodiments, the antigen-binding fragment can be substantially free of a full Fc domain. In some embodiments, the antigen-binding fragment can include a portion of a full Fc domain (e.g., CH2 or CH3 domain or a portion thereof). In some embodiments, the antigen-binding fragment can include a full Fc domain. In some embodiments, the Fc domain is an IgG domain, e.g., an IgG1, IgG2, IgG3, or IgG4 Fc domain. In some embodiments, the Fc domain comprises a CH2 domain and a CH3 domain.

As used herein, “administering” and similar terms mean delivering the composition to an individual being treated. Preferably, the compositions of the present disclosure are administered by, e.g., parenteral, including subcutaneous, intramuscular, or preferably intravenous routes.

As used herein, an “effective amount” means the amount of bioactive agent or diagnostic agent that is sufficient to provide the desired local or systemic effect at a reasonable risk/benefit ratio as would attend any medical treatment or diagnostic test. This can vary depending on the patient, the disease, the treatment being effected, and the nature of the agent. A therapeutically effective amount can vary depending upon the patient and disease condition being treated, the weight and age of the patient, the severity of the disease condition, the manner of administration and the like, which can readily be determined by one of ordinary skill in the art. The dosages for administration can range from, for example, about 1 ng to about 10,000 mg, about 5 ng to about 9,500 mg, about 10 ng to about 9,000 mg, about 20 ng to about 8,500 mg, about 30 ng to about 7,500 mg, about 40 ng to about 7,000 mg, about 50 ng to about 6,500 mg, about 100 ng to about 6,000 mg, about 200 ng to about 5,500 mg, about 300 ng to about 5,000 mg, about 400 ng to about 4,500 mg, about 500 ng to about 4,000 mg, about 1 μg to about 3,500 mg, about 5 μg to about 3,000 mg, about 10 μg to about 2,600 mg, about 20 μg to about 2,575 mg, about 30 μg to about 2,550 mg, about 40 μg to about 2,500 mg, about 50 μg to about 2,475 mg, about 100 μg to about 2,450 mg, about 200 μg to about 2,425 mg, about 300 μg to about 2,000, about 400 μg to about 1,175 mg, about 500 μg to about 1,150 mg, about 0.5 mg to about 1,125 mg, about 1 mg to about 1,100 mg, about 1.25 mg to about 1,075 mg, about 1.5 mg to about 1,050 mg, about 2.0 mg to about 1,025 mg, about 2.5 mg to about 1,000 mg, about 3.0 mg to about 975 mg, about 3.5 mg to about 950 mg, about 4.0 mg to about 925 mg, about 4.5 mg to about 900 mg, about 5 mg to about 875 mg, about 10 mg to about 850 mg, about 20 mg to about 825 mg, about 30 mg to about 800 mg, about 40 mg to about 775 mg, about 50 mg to about 750 mg, about 100 mg to about 725 mg, about 200 mg to about 700 mg, about 300 mg to about 675 mg, about 400 mg to about 650 mg, about 500 mg, or about 525 mg to about 625 mg of an antibody or antigen binding portion thereof, as provided herein. Dosing may be, e.g., every week, every 2 weeks, every three weeks, every 4 weeks, every 5 weeks or every 6 weeks. Dosage regimens may be adjusted to provide the optimum therapeutic response. An effective amount is also one in which any toxic or detrimental effects (side effects) of the agent are minimized and/or outweighed by the beneficial effects. Administration may be intravenous at exactly or about 3 mg/kg, 6 mg/kg, 10 mg/kg, 12 mg/kg or 24 mg/kg, at a frequency of weekly (once every week) or biweekly (once every 2 weeks). Additional dosing regimens are described below.

As used herein, “pharmaceutically acceptable” shall refer to that which is useful in preparing a pharmaceutical composition that is generally safe, non-toxic, and neither biologically nor otherwise undesirable and includes that which is acceptable for veterinary use as well as human pharmaceutical use. Examples of “pharmaceutically acceptable liquid carriers” include water and organic solvents. Preferred pharmaceutically acceptable aqueous liquids include PBS, saline, and dextrose solutions etc.

Various aspects of the disclosure are described in further detail below. Additional definitions are set out throughout the specification.

### Non-Depleting B Cell Inhibitors and Pharmaceutical Compositions

In various embodiments, a B cell inhibitor can be used to treat SLE and other autoimmune or allergic diseases. In some embodiments, such B cell inhibitors are non-depletional immunomodulators. As used herein, “non-depletional” or “non-depleting” means that the inhibitor or immunomodulator does not completely deplete B cell activities. On the other hand, “depletion” of B cells means that the agent acts to eliminate or destroy B cells, such as anti-CD20 antibodies, e.g., Rituximab. Thus, in one embodiment, the non-depletional B cell inhibitors or immunomodulators disclosed herein are not Rituximab. In some embodiments, the non-depletional B cell inhibitors or immunomodulators are not anti-CD20 antibodies or other CD20 inhibitors.

Exemplary non-depletional B cell inhibitors include, but are not limited to, CD32B×CD79B bi-specific inhibitors; CD32B modulators; B cell receptor (BCR) blockers, e.g., anti-CD22 molecules; B cell survival and activation inhibitors, e.g., B-cell activating factor (BAFF) or A proliferation-inducing ligand (APRIL) inhibitors such as belimumanb; anti-CD40 and anti-CD40L molecules; and Bruton's tyrosine kinase (BTK) inhibitors such as Ibrutinib (PCI-32765) and Acalabrutinib.

In some embodiments, the B cell inhibitor can be a CD32B×CD79B bi-specific antibody such as those disclosed in U.S. Publication No. 2016/0194396, WIPO Publication Nos. WO 2015/021089, and WO2017/214096, all incorporated by reference in its entirety, or an antigen-binding fragment thereof.

An exemplary CD32B×CD79B bispecific diabody can comprise two or more polypeptide chains, and can comprise:

(1) a VL Domain of an antibody that binds CD32B
(VL_{CD32B}), such VL_{CD32B} Domain having the sequence
(SEQ ID NO: 1):
DIQMTQSPSS LSASVGDRVT ITCRASQEIS GYLSWLQQKP
GKAPRRLIYA ASTLDSGVPS RFSGSESGTE FTLTISSLQP
EDFATYYCLQ YFSYPLTFGG GTKVEIK
(2) A VH Domain of an antibody that binds CD32B
(VH_{CD32B}), such VH_{CD32B} Domain having the sequence
(SEQ ID NO: 2):
EVQLVESGGG LVQPGGSLRL SCAASGFTFS DAWMDWVRQA
PGKGLEWVAE IRNKAKNHAT YYAESVIGRF TISRDDAKNS
LYLQMNSLRA EDTAVYYCGA LGLDYWGQGT LVTVSS
(3) A VL Domain of an antibody that binds CD79B
(VL_{CD79B}), such VL_{CD79B} Domain having the sequence
(SEQ ID NO: 3):
DVVMTQSPLS LPVTLGQPAS ISCKSSQSLL DSDGKTYLNW
FQQRPGQSPN RLIYLVSKLD SGVPDRFSGS GSGTDFTLKI
SRVEAEDVGV YYCWQGTHFP LTFGGGTKLE IK
(4) A VH Domain of an antibody that binds CD79B
(VH_{CD79B}), such VH_{CD79B} Domain having the sequence
(SEQ ID NO: 4):
QVQLVQSGAE VKKPGASVKV SCKASGYTFT SYWMNWVRQA
PGQGLEWIGM IDPSDSETHY NQKFKDRVTM TTDTSTSTAY
MELRSLRSDD TAVYYCARAM GYWGQGTTVT VSS

In one embodiment, the B cell inhibitor can be PRV-3279, a humanized CD32B×CD79B DART® protein produced in Chinese hamster ovary cells with a molecular weight of 111.5 kDa. DART® proteins are bispecific, antibody-based molecules that can bind 2 distinct antigens simultaneously. PRV-3279 is designed to target CD32B (Fc gamma receptor IIb) and CD79B (immunoglobulin-associated beta subunit of the B cell receptor (BCR) complex) on B lymphocytes. Co-ligation of CD32B and CD79B in preferential cis-binding mode on B lymphocytes triggers CD32B-coupled immunoreceptor tyrosine-based inhibitory motif signaling, which decreases antigen-mediated naïve and memory B cell activation without broad depletion. To prolong in vivo half-life, PRV-3279 also contains a human immunoglobulin G (IgG)1 Fc region that has been mutated to greatly reduce or eliminate undesired binding to FcγRs and complement but retains affinity for the neonatal FcR binding to take advantage of the IgG salvage pathway mediated by this receptor.

The CD32B molecule is a transmembrane inhibitory receptor expressed widely on B cells and other immune effector cells such as macrophages, neutrophils, and mast cells. The anti-CD32B component of PRV-3279 is based on a humanized version of MacroGenics' proprietary murine monoclonal antibody (mAb) 8B5. CD79B is an essential signal transduction component of the BCR that is expressed exclusively on B cells. The anti-CD79B component of PRV-3279 is based on a humanized version of the murine mAb CB3.

In one embodiment, PRV-3279 comprises the following sequence (the CDRs are underlined and coil domains are in bold):

Chain1 (Fc - CD32BVL - CD79bVH - E coil):
(SEQ ID NO.: 5)
DKTHTCPPCPAPEAAGGPSVFLFPPKPKDTLMISRTPEVTCVVVDVSHEDP
EVKFNWYVDGVEVHNAKTKPREEQYNSTYRVVSVLTVLHQDWLNGKEYKCK
VSNKALPAPIEKTISKAKGQPREPQVYTLPPSREEMTKNQVSLWCLVKGFY
PSDIAVEWESNGQPENNYKTTPPVLDSDGSFFLYSKLTVDKSRWQQGNVFS
CSVMHEALHNHYTQKSLSLSPGKAPSSSPMEDIQMTQSPSSLSASVGDRVT
ITCRASQEISGYLSWLQQKPGKAPRRLIYAASTLDSGVPSRFSGSESGTEF
TLTISSLQPEDFATYYCLQYFSYPLTFGGGTKVEIKGGGSGGGGQVQLVQS
GAEVKKPGASVKVSCKASGYTFTSYWMNWVRQAPGQGLEWIGMIDPSDSET
HYNQKFKDRVTMTTDTSTSTAYMELRSLRSDDTAVYYCARAMGYWGQGTTV
TVSSGGCGGGEVAALEKEVAALEKEVAALEKEVAALEKGGGNS
Chain2 (CD79bVL - CD32BVH - K coil):
(SEQ ID NO.: 6)
DVVMTQSPLSLPVTLGQPASISCKSSQSLLDSDGKTYLNWFQQRPGQSPNR
LIYLVSKLDSGVPDRFSGSGSGTDFTLKISRVEAEDVGVYYCWQGTHFPLT
FGGGTKLEIKGGGSGGGGEVQLVESGGGLVQPGGSLRLSCAASGFTFSDAW
MDWVRQAPGKGLEWVAEIRNKAKNHATYYAESVIGRFTISRDDAKNSLYLQ
MNSLRAEDTAVYYCGALGLDYWGQGTLVTVSSGGCGGGKVAALKEKVAALK
EKVAALKEKVAALKE
Chain3 (Fc):
(SEQ ID NO.: 7)
DKTHTCPPCPAPEAAGGPSVFLFPPKPKDTLMISRTPEVTCVVVDVSHEDP
EVKFNWYVDGVEVHNAKTKPREEQYNSTYRVVSVLTVLHQDWLNGKEYKCK
VSNKALPAPIEKTISKAKGQPREPQVYTLPPSREEMTKNQVSLSCAVKGFY
PSDIAVEWESNGQPENNYKTTPPVLDSDGSFFLVSKLTVDKSRWQQGNVFS
CSVMHEALHNRYTQKSLSLSPGK

In some embodiments, the pharmaceutical composition comprises a B cell inhibitor as disclosed herein and a pharmaceutically acceptable carrier. The B cell inhibitor can be formulated with the pharmaceutically acceptable carrier into a pharmaceutical composition.

As used herein, “pharmaceutically acceptable carrier” includes any and all solvents, dispersion media, coatings, antibacterial and antifungal agents, isotonic and absorption delaying agents, buffers, and other excipients that are physiologically compatible. Preferably, the carrier is suitable for parenteral, oral, or topical administration. Depending on the route of administration, the active compound, e.g., small molecule or biologic agent, may be coated in a material to protect the compound from the action of acids and other natural conditions that may inactivate the compound.

Pharmaceutically acceptable carriers include sterile aqueous solutions or dispersions and sterile powders for the extemporaneous preparation of sterile injectable solutions or dispersion, as well as conventional excipients for the preparation of tablets, pills, capsules and the like. The use of such media and agents for the formulation of pharmaceutically active substances is known in the art. Except insofar as any conventional media or agent is incompatible with the active compound, use thereof in the pharmaceutical compositions provided herein is contemplated. Supplementary active compounds can also be incorporated into the compositions.

A pharmaceutically acceptable carrier can include a pharmaceutically acceptable antioxidant. Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and nonaqueous carriers which may be employed in the pharmaceutical compositions provided herein include water, ethanol, polyols (such as glycerol, propylene glycol, polyethylene glycol, and the like), and suitable mixtures thereof, and injectable organic esters, such as ethyl oleate. When required, proper fluidity can be maintained, for example, by the use of coating materials, such as lecithin, by the maintenance of the required particle size in the case of dispersions, and by the use of surfactants. In many cases, it may be useful to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

These compositions may also contain functional excipients such as preservatives, wetting agents, emulsifying agents and dispersing agents.

Therapeutic compositions typically must be sterile, non-phylogenic, and stable under the conditions of manufacture and storage. The composition can be formulated as a solution, microemulsion, liposome, or other ordered structure suitable to high drug concentration.

Sterile injectable solutions can be prepared by incorporating the active compound in the required amount in an appropriate solvent with one or a combination of ingredients enumerated above, as required, followed by sterilization, e.g., by microfiltration. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation include vacuum drying and freeze-drying (lyophilization) that yield a powder of the active ingredient plus any additional desired ingredient from a previously sterile-filtered solution thereof. The active agent(s) may be mixed under sterile conditions with additional pharmaceutically acceptable carrier(s), and with any preservatives, buffers, or propellants which may be required.

Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation.

Exemplary dosage ranges for administration of an antibody include: 10-1000 mg (antibody)/kg (body weight of the patient), 10-800 mg/kg, 10-600 mg/kg, 10-400 mg/kg, 10-200 mg/kg, 30-1000 mg/kg, 30-800 mg/kg, 30-600 mg/kg, 30-400 mg/kg, 30-200 mg/kg, 50-1000 mg/kg, 50-800 mg/kg, 50-600 mg/kg, 50-400 mg/kg, 50-200 mg/kg, 100-1000 mg/kg, 100-900 mg/kg, 100-800 mg/kg, 100-700 mg/kg, 100-600 mg/kg, 100-500 mg/kg, 100-400 mg/kg, 100-300 mg/kg, and 100-200 mg/kg. Exemplary dosage schedules include once every three days, once every five days, once every seven days (i.e., once a week), once every 10 days, once every 14 days (i.e., once every two weeks), once every 21 days (i.e., once every three weeks), once every 28 days (i.e., once every four weeks), once a month, once every 5 weeks, and once every 6 weeks.

In some embodiments, an about 5-40 mg/kg, about 5-20 mg/kg or about 10 mg/kg per dose of PRV-3279 can be administered once every 2 weeks, once every 3 weeks, once every 4 weeks, once every 5 weeks 5 or once every 6 weeks. One or more doses can be administered, such as 1 dose, 2 doses or 3 doses. Administration can be via IV infusion. Any combination of the foregoing (e.g., 3 doses of 10 mb/kg per dose, once every 4 weeks) can be used. In some embodiments, the first dose can be given 2-6 weeks (e.g., 4 weeks) before gene therapy, the second dose at around the same time of the gene therapy, and the third dose 2-6 weeks (e.g., 4 weeks) after gene therapy. Thereafter, the patient can be monitored by examining the amount of specific antibodies against gene therapy vector (e.g., rAAV) and/or the transgene. If no or little antibody can be detected, then there can be no need for additional PRV-3279. If significant amount of antibody is present, then one or more dose of PRV-3279 can be administered.

It may be advantageous to formulate parenteral compositions in unit dosage form for ease of administration and uniformity of dosage. Unit dosage form as used herein refers to physically discrete units suited as unitary dosages for the patients to be treated; each unit contains a predetermined quantity of active agent calculated to produce the desired therapeutic effect in association with any required pharmaceutical carrier. The specification for unit dosage forms are dictated by and directly dependent on (a) the unique characteristics of the active compound and the particular therapeutic effect to be achieved, and (b) the limitations inherent in the art of compounding such an active compound for the treatment of sensitivity in individuals.

Actual dosage levels of the active ingredients in the pharmaceutical compositions disclosed herein may be varied so as to obtain an amount of the active ingredient which is effective to achieve the desired therapeutic response for a particular patient, composition, and mode of administration, without being toxic to the patient. “Parenteral” as used herein in the context of administration means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection, and infusion.

The phrases “parenteral administration” and “administered parenterally” as used herein refer to modes of administration other than enteral (i.e., via the digestive tract) and topical administration, usually by injection or infusion, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection, and infusion. Intravenous injection and infusion are often (but not exclusively) used for antibody administration.

When agents provided herein are administered as pharmaceuticals, to humans or animals, they can be given alone or as a pharmaceutical composition containing, for example, 0.001 to 90% (e.g., 0.005 to 70%, e.g., 0.01 to 30%) of active ingredient in combination with a pharmaceutically acceptable carrier.

### Therapeutic Uses and Methods

The compositions disclosed herein can be used to prevent, intercept, and treat autoimmune diseases mediated by B cells and/or autoantibodies. In some embodiments, the disease can be selected from Systemic Lupus Erythematosus (SLE), Multiple Sclerosis (MS), Rheumatoid Arthritis (RA), Psoriasis, Dermatomyositis/Polymyositis, Sjogren's Syndrome (SS), Primary Vasculitis (e.g. Polymyalgia rheumatica/Giant cell arteritis/Behçets), Graft vs. Host Disease (GVHD), Myasthenia Gravis, Pemphigus, Neuromyelitis Optica, Anti-NMDA receptor encephalitis, Guillain-Barré syndrome, Chronic inflammatory demyelinating polyneuropathy (CIDP), Grave's opthalmopathy, IgG4 Related Disease (IgG4-RD), Idiopathic thrombocytopenic purpura (ITP), Inflammatory Bowel Disease (IBD), and Crohn's Disease. In some embodiments, the disease is Systemic Lupus Erythematosus.

B cell activation is not only prevalent, but also central to the pathogenesis of SLE (Zhang 2001; Stohl 2003; Chu 2009), supporting the rationale for down modulation of B cells in this disease. Given the need for chronic, sometimes lifelong immunosuppression in lupus, a particularly attractive B-cell-targeting therapeutic would be one that can rapidly inhibit all subsets of activated B cells but spare resting B cells from depletion or inactivation.

In some embodiments, PRV-3279 prevents flare, i.e., maintains the improvement in SLE signs and symptoms for 24 weeks following the amelioration of active disease induced by steroid treatment at baseline and after the withdrawal of major background medications. This can be measured by: (1) Investigator's assessment that SLE disease meets the Lupus Foundation of America (LFA) international consensus definition for flare with significant worsening on Clinician's Global Impression of Change (CGIC); (2) an increase from baseline of the hybrid Systemic Lupus Erythematosus Disease Activity Index (SLEDAI) ≥4 points; (3) ≥1 British Isles Lupus Assessment Group (BILAG) A; and/or (4) BILAG B score with a rating of “worse” or “new.”

In some embodiments, PRV-3279 prolongs duration of disease amelioration initiated by corticosteroids (for example, intramuscular (IM) injection of methylprednisolone acetate (Depo-Medrol® or equivalent) 20-100 mg), in the absence of concomitant medication with the exception of antimalarials, up to 10 mg prednisone (or equivalent corticosteroid) and non-steroidal anti-inflammatory drugs (NSAIDs).

In some embodiments, PRV-3279 allows patients to achieve and sustain low dose corticosteroids. In some embodiments, PRV-3279 allows patients to achieve and sustain European League Against Rheumatism (EULAR)-recommended treatment goals of both low disease and low steroids. In some embodiments, PRV-3279 improves patient-reported rating of their physical functioning, and reduces one or more signs and symptoms of SLE using a stringent definition for improvement in each symptom based upon the SLE Responder Index-4 (SRI-4). In some embodiments, PRV-3279 reduces disease activity in all organs that were rated as moderate or severely active at baseline by the British Isles Lupus Assessment Group (BILAG) Index.

In other embodiments, the effects of PRV-3279 are associated with certain pre-defined phenotypes such as the B cell or Plasma cells signature, and the/or absence of Inflammatory/Type 1 interferon signature, in peripheral blood mRNA analyses.

### EXAMPLES

The following examples, including the experiments conducted and results achieved, are provided for illustrative purposes only and are not to be construed as limiting the disclosure.

### Example 1: Safety and Tolerability of PRV-3279

The multiple ascending dose (MAD) study (PREVAIL 1), was designed to assess the safety and tolerability of PRV-3279, which is given as three infusions at two dose levels (3 and 10 mg/kg) administered once every two weeks. Secondary objectives were to characterize the multidose PK and the immunogenicity of PRV-3279. Exploratory objectives included exploration of the effects of PRV-3279 on potential biomarkers for target engagement and B-cell function.

Sixteen healthy subjects were enrolled. Each cohort of 8 subjects received PRV-3279 or placebo at a ratio of 3:1 (n=6 for PRV-3279 and n=2 for placebo). Fourteen subjects received all planned treatments per protocol and completed the study Clinical Safety Results.

PRV-3279 was well tolerated. There were no adverse events of special interest (AESIs), serious treatment emergent adverse events (TEAEs), serious adverse events (SAEs), or TEAEs leading to death. One (16.7%) subject who received PRV-3279 10 mg/kg had 4 mild TEAEs considered by the Investigator to be related to the study drug (abdominal pain, feeling hot, cold sweat, and hyperhidrosis) and also withdrew from the study due to these adverse events. A total of 34 TEAEs were reported in 9 (56.3%) subjects. The most frequently reported TEAEs excluding catheter or venipuncture site adverse events were feeling hot (3 TEAEs) and cold sweat (2 TEAEs each). All other TEAEs were reported once each. Two TEAEs were moderate in severity, and the remainder were mild. All out-of-range clinical laboratory values, vital signs measurements, ECG results, and physical examination findings were evaluated as not related to the study drug and not clinically significant; none were reported as TEAEs.

### Pharmacokinetic Results:

The t1/2 on Day 29 was 157 h (6.54 days) and 185 h (7.71 days) for the 3 mg/kg and 10 mg/kg dose levels, respectively. The volume of distribution at steady state (Vss) was comparable for the 3 mg/kg and 10 mg/kg doses (618 mL/kg and 576 mL/kg). CL on Day 29 was slightly lower for the 10 mg/kg dose (1.63 mL/h/kg) than for the 3 mg/kg dose (2.71 mL/h/kg).

After multiple dosing to Day 29, there was minimum accumulation of PRV-3279 as shown by accumulation ratios based on Cₘₐₓ (RacCₘₐₓ) and AUC (RacAUC₀₋₃₃₆) values for the 3 and 10 mg/kg doses of 1.08 and 1.25 and 1.33 and 1.49 levels, respectively. The geometric mean (GM) Vₛₛ on Day 29 was comparable for the 3 mg/kg and 10 mg/kg dose levels (61.8 mL/kg and 57.6 mL/kg). The GM CL on Day 29 was lower for the 10 mg/kg dose (0.163 mL/h/kg) compared to the 3 mg/kg dose (0.271 mL/h/kg).

### Immunogenicity Results:

Consistent with the mechanism of action and ability of PRV-3279 to inhibit its own immunogenicity, incidence of ADA was higher at 3 mg/kg (6 out of 6) compared to 10 mg/kg (4 out of 6) at the end of study. The assay used was validated and drug tolerant (not affected by the presence of PRV-3279). The ADA titers were generally low. Although the numbers of subjects per cohort provide a small sample size, there was no apparent effect of ADA on PK variables. Evaluation of the mean serum concentrations of PRV-3279 by ADA results shows a trend for slightly higher concentrations up to about Day 43 at the 3 mg/kg dose when ADA are positive. No trend can be identified for the 10 mg/kg dose due to limited data. The number of ADA-positive subjects increased over time. At the 3 mg/kg dose level, the first subject with a positive result was on Day 15. All 6 subjects of this dose level tested positive on Day 85. At the 10 mg/kg dose level, the first subject who tested ADA positive was on Day 36. On Day 85, 4 of the 6 subjects were ADA positive.

### Pharmacodynamic Results:

After initial dosing with 3 and 10 mg/kg PRV-3279, >85% total number of available CD19+ B cells were bound. The binding pattern was similar for memory B cells (CD19+/CD27+) and naïve B cells (CD19+/CD27−). Binding intensity to B cells by PRV-3279 did not differ in different B cell sub-populations and slightly higher for 10 mg/kg than 3 mg/kg, At the 10 mg/kg dose, levels of >50% receptor occupancy, which is considered the minimum level of binding required for optimal B-cell modulation, were detected for up to 28 days after the final dose. Both groups declined to approximately baseline levels at Day 85. Due to sample instability, these percent binding values were likely underestimated.

The binding of B cells was associated with a functional decrease in IgM production of −35% to −44% in actively treated subjects, which persisted until the end of study. There was a dose-response trend, with the 10 mg/kg achieving greater reductions in IgM and also showing a reduction in IgE. No drug effect was observed on IgG levels. This was expected given that there was no concurrent antigen stimulus and IgG half-life is longer than the other classes. Peripheral B cell counts showed short-term reduction of <50%, and no abnormalities were seen in other immune cell types. No cytokine release was measurable after dosing with PRV-3279.

### Example 2: Inhibition of B Cell Function by PRV-3279

In some embodiments, the B cell inhibitor PRV-3279 given three times every 2 weeks at 3 mg/kg IV and every 2 weeks at 10 mg/kg IV resulted in profound and durable inhibition of B cell function in healthy volunteers.

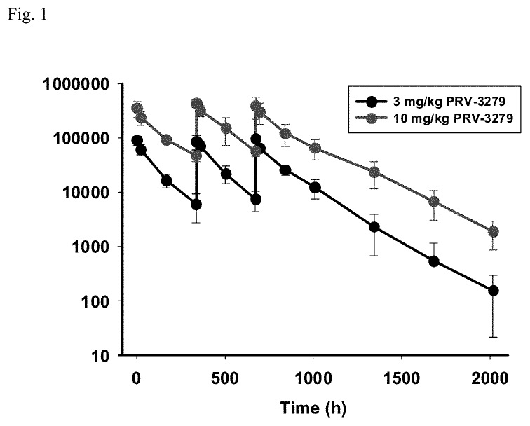

Pharmacokinetic parameters were generally dose-proportional between the 3 mg/kg and 10 mg/kg groups with little accumulation upon 2qw repeat dosing (FIG. 1). The half-life of the drug was observed to be around 1 week after repeat dosing for both the 3 mg/kg and 10 mg/kg dose levels (Table 1).

TABLE 1
Summary of PRV-3279 Pharmacokinetic
Parameters by Dose and Day (Geometric
Mean {% CV)) 3 mg/kg (n = 6) 10 mg/kg (n = 6)

| Parameter | Day 1 | | Day 29 | | Day 1 | | Day 29 * | |
|---|---|---|---|---|---|---|---|---|
| Cmax | 90.6 | (21.0) | 97.9 | (14.4) | 346 | (3 4.7) | 461 | (13.6) |
| (μg/ml) | | | | | | | | |
| AUCo-335 | 348 | (23.7) | 463 | {1 4.3) | 1688 | (14.2) | 2563 | (11.5) |
| (μg-d/ml) | | | | | | | | |
| Half- | 3.85 | (19.5) | 6.54 | (18.6) | 5.25 | (13.8) | 7.71 | (13.3) |
| Life (d) | | | | | | | | |
| Cl | | | 2.71 | (14.3) | | | 1.63 | (11.5) |
| (mL/h/kg) | | | | | | | | |
| Vss (L/kg) | | | 0.618 | (23.6) | | | 0.576 | (26.8) |

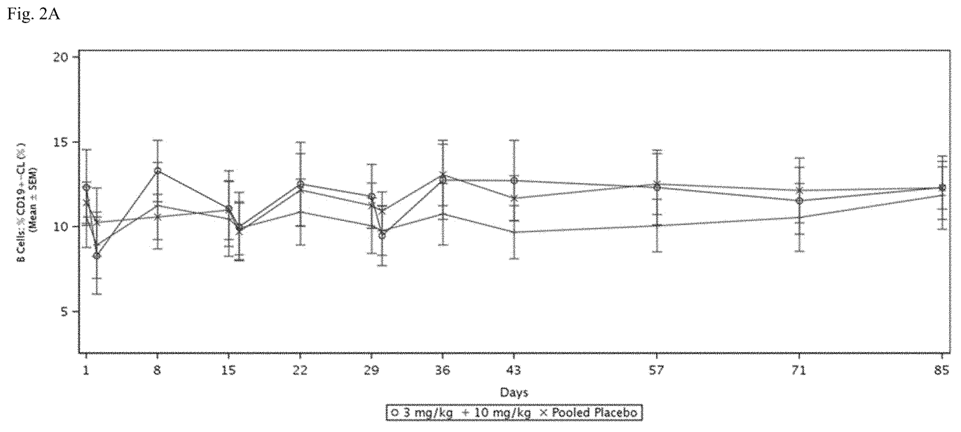

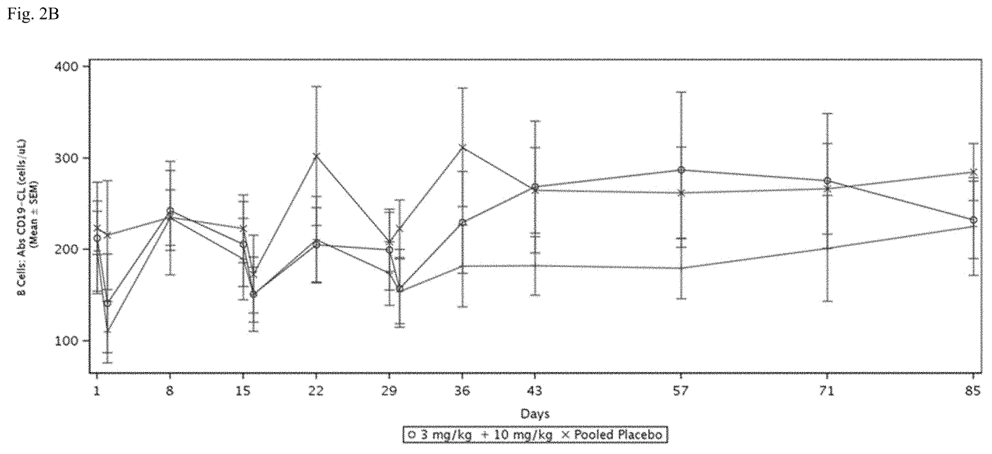

As shown in FIGS. 2A-2B, no sustained depletion of B cells or other leukocyte populations was observed after repeat dosing of 3 mg/kg or 10 mg/kg. Transient decrease in peripheral B cell counts were observed which was less pronounced after second and third dose of PRV-3279 and quickly recovered after each dose.

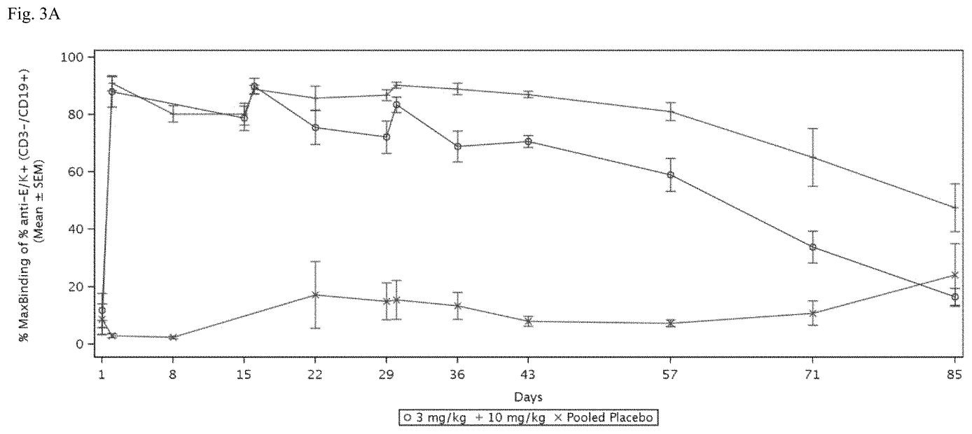

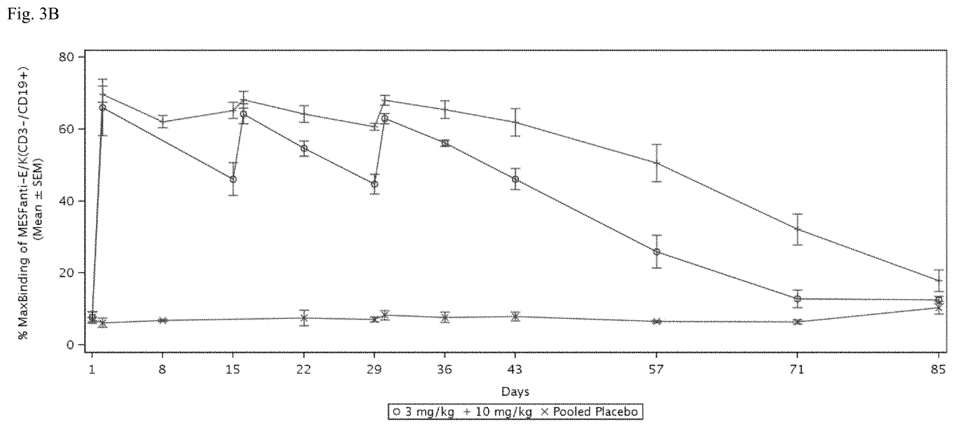

Administration of PRV-3279 resulted in dose-proportional, extensive and sustained binding to circulating B lymphocytes. PRV-3279 bound >85% of available B cells, memory B cells and naïve B cells after dosing, which was maintained for up to 2 months after repeat dosing at 10 mg/kg (FIG. 3A). Approximately 70 to 75% of receptors were occupied by drug on B cells, memory B cells, and naïve B cells, whereby 10 mg/kg of PRV-3279 maintained >50% receptor binding for up to 28 days after the final dose (FIG. 3B). Of note, previous in vitro studies have demonstrated that PRV-3279 mediated inhibitory function of B cells reaches optimal effect when over 50% of maximal PRV-3279 binding to human B cells is achieved.

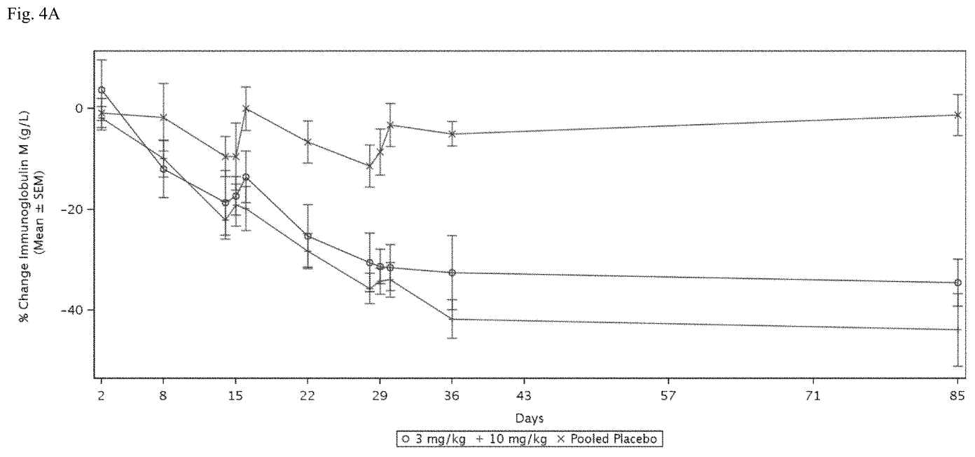

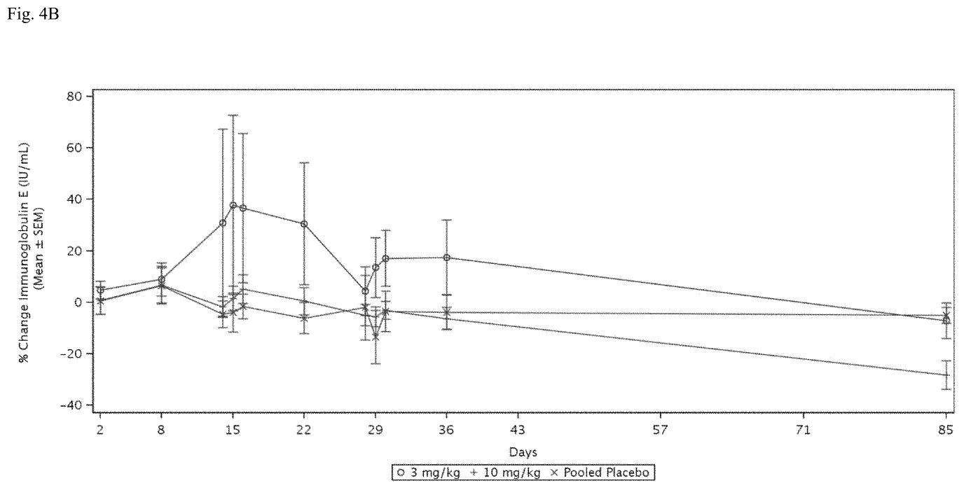

An extended pharmacodynamic effect, consistent with the inhibition of B cell function was demonstrated by the reduction in circulating immunoglobulin M levels (FIG. 4A) and a later reduction in IgE levels (FIG. 4B). For IgM this effect was dose dependent with a maximal inhibition of around 44% observed at 10 mg/kg after repeat dosing, which was sustained for at least 8 weeks after the final dose. IgE was reduced by up to 30% after the final dose of 10 mg/kg.

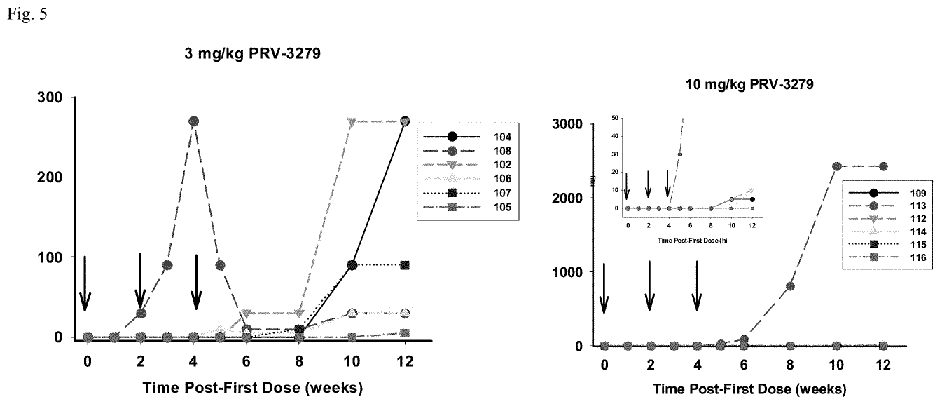

Anti-drug antibody (ADA) production was observed at both dose levels tested (FIG. 5). However, consistent with the mechanism of action of B cell inhibition it was dose and time dependent. Immunogenicity was found not to affect exposure, safety or pharmacodynamic parameters.

There was a well-tolerated safety profile. There were no AESIs, serious TEAEs, SAEs, or TEAEs leading to death. One (16.7%) PRV-3279 10 mg/kg subject experienced 4 mild TEAEs considered by the Investigator to be related to the study drug (abdominal pain, feeling hot, cold sweat, and hyperhidrosis) that led to discontinuation.

TABLE 2
Summary of PRV-3279 safety by system organ class/preferred term

| | PRV-3279 | PRV-3279 | Pooled |
|---|---|---|---|
| | 3 mg/kg | 10 mg/kg | Placebo |
| System Organ | (N = 6) | (N = 6) | (N = 4) |
| Class/Preferred Term | n (%) E | n (%) E | n (%) E |
| Number of subjects and events | 3 (50.0) 12 | 5 (83.3) 18 | 1 (25.0) 4 |
| with at least 1 TEAE | | | |
| Number of subjects and events | 0 | 0 | 0 |
| with SAEs | | | |
| General disorders and | 3 (50.0) 6 | 2 (33.3) 4 | 1 (25.0) 3 |
| administration site conditions | | | |
| Skin and subcutaneous tissue | 1 (16.7) 2 | 3 (50.0) 6 | 0 |
| disorders | | | |
| Nervous system disorders | 1 (16.7) 1 | 1 (16.7) 1 | 1 (25.0) 1 |
| Gastrointestinal disorders | 1 (16.7) 1 | 1 (16.7) 2 | 0 |
| Respiratory, thoracic and | 0 | 2 (33.3) 2 | 0 |
| mediastinal disorders | | | |
| Ear and labyrinth disorders | 0 | 1 (16.7) 1 | 0 |
| Infections and infestations | 0 | 1 (16.7) 1 | 0 |
| Injury, poisoning and procedural | 1 (16.7) 1 | 0 | 0 |
| complications | | | |
| Musculoskeletal and connective | 1 (16.7) 1 | 0 | 0 |
| tissue disorders | | | |
| Reproductive system and breast | 0 | 1 (16.7) 1 | 0 |
| disorders | | | |

### Example 3: Ex Vivo Study of Inhibitory Activity of PRV-3279

The inhibitory activity of PRV-3279 towards B cells was compared in B cells isolated from whole blood samples obtained from either normal healthy volunteers or patients with SLE with varying degrees of disease severity as defined by the SLE Disease Activity Index. Patients were defined as inactive/mild or active, respectively. Briefly, purified B cells (5×10⁴/well) from normal healthy subjects or patients with SLE were incubated with 100 nM PRV-3279 for 30 minutes in a 96-well tissue culture plate and then stimulated with anti-human IgM antibody (anti-0 at 10 μg/mL for 48 hours An in vitro 3H-thymidine incorporation B-cell proliferation assay was then used to evaluate PRV-3279 activity in these samples.

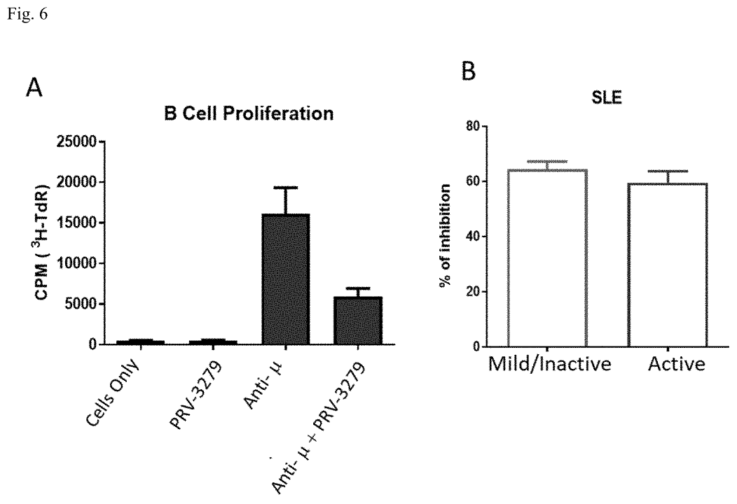

As shown in FIG. 6, the B cell inhibitor inhibits human B cell proliferation in healthy and Lupus subjects. That inhibition is evidenced ex-vivo, whereby PRV-3279 inhibited both normal healthy volunteers and SLE patient samples to a similar degree (60%). Furthermore, PRV-3279 inhibited B-cell proliferation by approximately 60% regardless of the active or inactive disease status in these SLE patient samples. This is surprising since CD32B signaling has been shown to be perturbed in some lupus patients and provides evidence of the potential for PRV-3279 activity in the setting of autoimmune pathological B cells.

### Example 4: Phase 2a, Randomized, Double-Blind, Placebo-Controlled Trial of PRV-3279 EVAluation In Lupus (PREVAIL-2)

Rationale: PRV-3279 is a humanized dual affinity re-targeting (DART®) protein that binds to both CD32B (Fcγ receptor IIb) and CD79B on B cells only. The mechanism of action and early clinical data suggest that PRV-3279 can be a safe and effective treatment for chronic systemic lupus erythematosus (SLE).

Objectives and Endpoints:

| | |
|---|---|
| Objectives | Endpoints |

Primary

| | |
|---|---|
| To evaluate the ability of PRV-3279 to | Proportion of patients who maintain the |
| prevent flare, i.e., to maintain the | improvement in SLE disease activity from |
| improvement in SLE signs and symptoms | Baseline (Day 1) to Week 24, defined as |
| for 24 weeks, after the amelioration of active | nolupus flare during this period. |
| disease induced by corticosteroid treatment | A lupus flare is defined as: |
| before Day 1 with the withdrawal of major | Investigator's assessment that the SLE |
| background medications. | activity meets the Lupus Foundation of |
| | America (LFA) international |
| | consensus definition for a flare¹ AND |
| | A score of “definite worsening” or “severe |
| | worsening” on Clinician's Global |
| | Impression of Change (CGIC), AND |
| | at least one of the following occurrences: |
| | an increase of ≥4 points from baseline |
| | in the hybrid Safety of Estrogens in |
| | Lupus Erythematosus National |
| | Assessment-Systemic Lupus |
| | Erythematosus Disease Activity Index |
| | (SELENA-SLEDAI) score |
| | (hSLEDAI), OR |
| | ≥1 organ with an A score (severe) or |
| | BScore (moderate) item rated new or |
| | worse on British Isles Lupus |
| | Assessment Group (BILAG) Index |

Secondary

| | |
|---|---|
| Efficacy: | |
| 1. To evaluate whether PRV-3279 prolongs | 1. Time to treatment failure. |
| the duration of disease amelioration | Treatment failure is defined compared |
| induced by corticosteroids before Day 1. | to Baseline (Day 1): |
| | Occurrence of an SLE flare (as |
| | defined in the primary endpoint), OR |
| | Missing 2 consecutive doses or 3 or |
| | more total doses of the study drug |
| | for any reason, OR |
| | Initiation of a new SLE medication, OR |
| | Increased dose of current SLE |
| | medication with the exception of |
| | nonsteroidal anti-inflammatory |
| | drugs(NSAIDs), OR |
| | Patient withdrawal from the study |
| | before the Week 24 visit |
| 2. To determine whether PRV-3279 allows | 2. Proportion of patients who meet either of |
| patients to achieve and sustain European | the following criteria at Week 24: |
| League Against Rheumatism (EULAR)- | hSLEDAI score <3 OR |
| recommended treatment goal of low | All BILAG scores are C or less |
| disease. | |
| 3. To evaluate the effect of PRV-3279 on | 3. Change from Screening to Week 24 in |
| patient-reported physical functioning. | the Physical Component Score (PCS) score |
| | in the Short Form 36 Health Survey (SF-36). |
| 4. To determine whether PRV-3279 can | 4. Proportion of patients who meet the criteria |
| reduce one or more signs and symptoms of | for SRI-4 at Week 24 compared to Screening. |
| SLE using a stringent definition for | SRI-4 is defined as: |
| improvement in each sign or symptom, | A hSLEDAI score decrease of ≥4 points, |
| based on the SLE Responder Index-4 | AND |
| (SRI-4) | No new organs with a BILAG A |
| | (severe)score, AND |
| | No more than 1 new organ with a |
| | BILAG B (moderate) score, AND |
| | No SELENA-SLEDAI Physician's |
| | Global Assessment (ssPGA) |
| | score increase of >0.3 points. |
| 5. To determine whether PRV-3279 reduces | 5. Proportion of patients who meet the |
| disease activity in all organs that are | BILAG-based Combined Lupus |
| rated as moderately or severely active at | Assessment (BICLA) criteria at Week 24 |
| Screening using the BILAG Index. | compared to Screening: |
| | Reduction by ≥1 grade in all organs |
| | with |
| | BILAG A or B scores AND |
| | No worsening of SLEDAI or |
| | other BILAG organs AND |
| | No ssPGA score increase of ≥0.3 |
| | points. |
| Safety: To evaluate the safety and | Frequency of treatment-emergent adverse |
| tolerability of PRV-3279. | events(TEAEs), serious adverse events |
| | (SAEs), TEAEs leading to drug withdrawal, |
| | adverse events of special interest (AESIs), |
| | and total serum immunoglobulin levels. |
| | Frequency of potentially clinically important |
| | changes in clinical laboratory tests, vital |
| | signs, electrocardiograms (ECGs), and |
| | physical examinations. |
| PK and Immunogenicity: To evaluate the | Serum concentrations of PRV- |
| pharmacokinetics (PK) and | 3279. Anti-drug antibody (ADA) |
| immunogenicity of PRV-3279 in patients | titers. |
| with SLE. | |

Exploratory

| | |
|---|---|
| 1. To determine whether the effects of PRV- | 1. Effect of elevated B cell gene signature |
| 3279 are associated with certain pre-defined | at Screening on efficacy endpoints after |
| phenotypes. | treatment with PRV-3279 |
| 2. To evaluate the pharmacodynamics | 2. Changes in clinical biomarkers, |
| (PD) of PRV-3279 in patients with SLE. | peripheral blood B cell lineage, and |
| | activation markers. |
| | Levels of CD32B/CD79B receptor |
| | occupancy on peripheral blood B cells (if |
| | performed). |
| | Correlation of these markers with PK of |
| | PRV-3279 and clinical response to PRV- |
| | 3279. |

¹A flare is a measurable increase in disease activity in one or more organ systems involving new or worse clinical signs and symptoms and/or laboratory measurements. It must be considered clinically significant by the assessor, and usually, there would be at least consideration of a change or an increase in treatment.

Study Design: This is a randomized, double-blind, placebo-controlled study in adult patients with active SLE. Approximately 100 eligible patients can be randomized at a 1:1 ratio to receive treatment with either 10 mg/kg PRV-3279 or placebo. The study drug, PRV-3279 or placebo, can be given as an intravenous (IV) infusion over 2 hours, every 4 weeks from Week 0 through Week 20 for a total of 6 doses. Two follow-up visits are planned (Week 24 and Week 28). The EOS visit can occur at Week 28.

During the Screening period, the patient can receive an intramuscular (IM) injection of methylprednisolone acetate (Depo-Medrol® or equivalent) at a dose of ≥40 mg to induce improvement of SLE signs and symptoms. Repeat injections may be given to further ameliorate symptoms, up to a total of 4 injections with a maximum total dose of 320 mg.

Starting on Day 1 (Baseline/Randomization visit) and continuing throughout the study, the only background SLE treatments that may be continued are hydroxychloroquine up to 400 mg per day (or other antimalarial), up to 10 mg per day prednisone (or equivalent corticosteroid), and NSAIDs. Patients can be asked to abstain from NSAIDs on the mornings of the Randomization visit and all subsequent study visits (NSAIDs can be re-started after completing all evaluations on the day of each study visit). On other days throughout the study, NSAIDs are allowed without restrictions. All other SLE treatments taken at the time of screening can be withdrawn during the Screening period and prior to Randomization.

Potentially eligible patients can return to the study site to confirm eligibility for randomization on Day 1. The Investigator must confirm that the patient has achieved at least a moderate improvement in SLE signs and symptoms as indicated by:
CGIC score of “definite improvement” or “major or complete improvement”
AND
≥4-point decrease in hSLEDAI score from Screening, OR improvement by ≥1 severity grade in at least one BILAG system that was severe (A score) or moderate (B score) at Screening (i.e., from A to B-D or from B to C or D).

Note that assessments by hSLEDAI and BILAG Index at the Randomization visit may not follow the standard 4-week assessment rules but can be scored by a simple clinical comparison of SLEdisease activity at Week 0 (Day 1, Randomization Visit) to the Screening Visit.

If the patient is confirmed eligible, randomization can occur through an interactive voice/webresponse system (IVRS/IWRS) and can be stratified by the presence or absence of serum anti-double-stranded deoxyribonucleic acid (anti-dsDNA) antibodies and the presence or absence of elevated B cell gene signature, as defined by B cell expression pathway testing.

At each visit, formal assessments of disease activity and safety can be conducted, and laboratory specimens can be collected. Patients can receive IV infusions of the study drug every 4 weeks from Week 0 through Week 20, inclusive.

All efforts should be made to retain all patients in the study, regardless of their compliance with study procedures. If a patient takes any new SLE medications (other than NSAIDs), increases the dose of current SLE medications (other than NSAIDs), misses 2 consecutive doses or 3 or more total doses of the study drug, then the patient should permanently discontinue study drug but continue study procedures and assessments, but can be documented as a nonresponder in the primary and applicable secondary efficacy endpoints.

If a lupus flare occurs (as defined in the primary endpoint), the patient should contact the study site immediately and be seen as soon as possible for a Flare Assessment visit, regardless of visit schedule, and should be assessed according to the Schedule of Activities (SoA, see Table 3). SLE medications may be prescribed as warranted to control the symptoms and/or signs present at this visit. If confirmed, the patient can discontinue any further study drug administration and can be considered a nonresponder in the primary and applicable secondary efficacy endpoints.

Throughout the study, SLE disease activity and patient-reported outcomes (PROs) can be assessed using the following instruments:

Disease Activity Instruments:
The hybrid Safety of Estrogens in Lupus National Assessment Systemic Erythematosus Lupus Erythematosus Disease Activity Index [hSLEDAI]
The SELENA-SLEDAI Physician's Global Assessment (ssPGA)
SELENA-SLEDAI Flare Index (SFI)
Modified SELENA-SLEDAI Flare Index (mSFI)
British Isles Lupus Assessment Group (BILAG) Index
Clinician's Global Impression of Change (CGIC)
Cutaneous Lupus Erythematous Disease Area and Severity Index (CLASI)
Tender and swollen joint counts

PROs:
Short Form 36 Health Survey (SF-36)
Patient Global Impression of Change in Clinical Status (PGIC)
Patient Global Impression of Change in Disease Severity (PGIS)
Functional Assessment of Chronic Illness Therapy-Fatigue (FACIT-Fatigue Scale)

Safety assessments can include TEAEs, vital signs, physical examination findings, 12-lead ECGs, and clinical laboratory tests (hematology, chemistry, urinalysis, coagulation panel, lupus-related serologies, SLE disease activity markers, and T cell, B cell, natural killer cell [TBNK] panel). A rapid COVID-19 test can be performed at Screening and prior to each study drug administration. A urine pregnancy test can be performed for women of childbearing potential (WOCBP) onsite prior to each study drug administration (serum pregnancy test at Screening).

PK (serum PRV-3279 concentrations), PD (biomarkers) and immunogenicity (ADAs) assessments can also be conducted.

Number of Patients: Approximately 100 patients can be randomized to receive study drug treatment. The study can be conducted at approximately 30 sites in United States (US) and Hong Kong. Approximately 50 patients can be assigned to each treatment group (PRV-3279 or placebo). Randomized patients who discontinue study drug or withdraw consent for study participation will not be replaced.

Treatment Groups and Duration:

Treatment Groups:
PRV-3279: 10 mg/kg IV infusion, administered over 2 hours, once every 4 weeks, from Week 0 to Week 20
Placebo: 0.9% sodium chloride IV infusion, administered over 2 hours, once every 4 weeks, from Week 0 to Week 20

Duration of Study Participation for Each Patient: 34 Weeks
Screening: up to 6 weeks
Study Treatment Period: 20 weeks
Follow-up: 8 weeks

Central Adjudication Committee (CAC): Study integrity can be supported with remote oversight by a CAC working in tandem with study clinical and data monitors to confirm entry qualification and accuracy of disease activity scoring and to adjudicate flares.

The CAC can consist of independent medical reviewers with clinical expertise in SLE. The CACresponsibilities can include:
Confirming patients' eligibility at Screening to enter the study
Adjudicating SLE flare
Ensuring the accuracy and consistency of scoring of the disease activity instruments

The CAC can be blinded to study treatment. Details of the CAC composition, objectives, and conduct can be described in the CAC charter.

Independent Data Monitoring Committee (IDMC): An IDMC consisting of 2 physicians and 1 statistician can be formed. Additionally, 1 or 2 external nonvoting advisors with experience in infectious disease, hematology, and other relevant subspecialties may be added as needed.

Throughout the study, the IDMC can continually review unblinded safety data. Meetings can beheld approximately quarterly. Details of the IDMC composition, objectives, and conduct can be described in the IDMC charter.

Statistical Methods: Details of the statistical methods can be provided in the Statistical Analysis Plan (SAP).

Primary efficacy analysis: The proportions of patients who meet the criteria of maintained improvement through Week 24 can be compared between the PRV-3279 and placebo groups using the Cochran-Mantel-Haenszel (CMH) test, accounting for randomization stratification factors of presence or absence of serum anti-dsDNA antibody and the presence or absence of elevated B cell gene signature.

The Full Analysis Set (FAS) of randomized patients can be used.

Secondary efficacy analysis:
1. The time to treatment failure can be summarized by treatment group using Kaplan-Meier analysis (median, 95% CI, number of events, number censored, etc.) and Kaplan-Meier plots. The stratified log-rank test can be used to test for difference between treatment groups. The proportion of patients who achieve the EULAR-recommended goal of low disease at Week 24 can be compared between the groups using the same test as the primary efficacy analysis.
2. The change from Screening to Week 24 in the SF-36 PCS can be analyzed using the Mixed Model for Repeated Measurements (MMRM). The model can include treatment, visit, randomization stratification factors, baseline score as fixed effects, and the treatment by visit as interaction term.
3. The proportion of patients who achieve SRI-4 at Week 24 can be compared between the groups using the same test as the primary efficacy analysis.
4. The proportions of patients who meet the BICLA criteria at Week 24 can be compared between the groups using the same test as the primary efficacy analysis.
5. The time to treatment failure in subgroups by stratification can be summarized by treatment using the Kaplan-Meier method and compared between treatment groups using the log-rank test.

Exploratory analysis: Details of the exploratory analysis can be provided in the SAP.

Safety analysis: TEAEs, SAEs, TEAEs leading to withdrawal of study drug, adverse events of special interest (AESIs), and other safety variables can be analyzed using descriptive statistics.

Immunogenicity: ADAs can be analyzed using descriptive statistics.

PK, PD, and PK/PD analyses: PK and PD data can be summarized. Other exploratory analyses, including the effect of CD32Bpolymorphisms on the response to PRV-3279, can be detailed in the SAP. Data from this study may be combined with other PK data for a more formal population PK analysis in a separate report.

Sample Size Determination: The published data from the Phase 2, double-blind, randomized, placebo-controlled study of a reversible B cell inhibitor, XmAb®5871, in SLE showed that the response rates of the maintenance improvement in SLE signs and symptoms in the intent-to-treat (ITT) population were 40.4% vs 23.1% at Day 225 and 57.7% vs 34.6% at Day 169 for the active arm and control arm, respectively. Assuming a similar treatment effect of PRV-3279 can be observed at Week 24 in this Phase 2a study, 45 patients in each treatment arm can provide at least 80% power to detect a 25% proportion difference (assuming the placebo response rate of 30%) between the PRV-3279 and placebo arms at a 0.20 significance level (2-sided). A total of 100 patients (50 patients in each treatment group) can be randomized to allow for up to 10% dropouts.

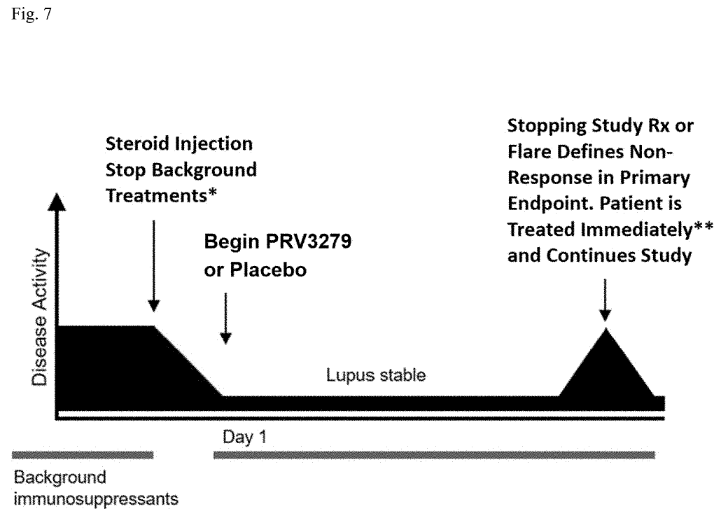

A schematic of the study is shown in FIG. 7. *All background SLE treatments are stopped or tapered after the Screening visit (before Day 1), except prednisone up to 10 mg daily (or equivalent corticosteroid), up to 400 mg per day hydroxychloroquine [or other antimalarial]), and/or NSAIDs. **At the time of a flare, the patient should be seen at the study site, regardless of scheduled visits.

TABLE 3
Schedule of Activities (SoA)

| | Period | | | | | | | | | | |
|---|---|---|---|---|---|---|---|---|---|---|---|
| | Screeing | Randomized Treatment Visits | | | | | | Follow-up | | | |
| | Week | | | | | | | | | | |
| | −6 to −1 | 0 | 4 | 8 | 12 | 16 | 20 | 24 | 28 (EOS) | | |
| | Day | | | | | | | | | | |
| | ≤−42 to −1 | 1 | 29 | 57 | 86 | 113 | 141 | 169 | 197 | Flare | |
| | | | | | | | | | | Assessment | |
| | | | | | | | | | | Visit¹ | ET Visit |
| | Visit Window (±days) | | | | | | | | | | |
| | | | ±3 | ±3 | ±3 | ±3 | ±3 | ±3 | ±3 | | +3 |
| Informed consent | X | | | | | | | | | | |
| Eligibility review | X | X | | | | | | | | | |
| Concomitant medications² | X | X | X | X | X | X | X | X | X | X | X |
| Complete medical history review | X | X | | | | | | | | | |
| Adverse events | X | X | X | X | X | X | X | X | X | X | X |
| Vital signs³ | X | X | X | X | X | X | X | X | X | X | X |
| Body weight | X | X | X | X | X | X | X | X | X | X | X |
| Height | X | | | | | | | | | | |
| Complete physical exam⁴ | X | | | | | | | | | | |
| Brief physical exam⁵ | X | X | X | X | X | X | X | X | X | X | X |
| Serum pregnancy test in WOCBP | X | | | | | | | | | | |
| Urine pregnancy test in WOCBP | | X | X | X | X | X | X | | | | |
| FSH & LH (Females - to confirm | X | | | | | | | | | | |
| nonsurgical menopause) | | | | | | | | | | | |
| Hematology, chemistry, coagulation, | X | X | X | X | X | X | X | X | X | X | X |
| urinalysis⁶ | | | | | | | | | | | |
| TBNK panel | X | X | X | X | X | X | X | X | X | X | X |
| HBV, HCV, HIV &TB tests⁷ | X | | | | | | | | | | |
| Rapid COVID-19 test | X | X | X | X | X | X | X | X | X | X | X |
| SLE serology tests⁸ | X | | | | X | | | X | X | | X |
| SLE disease activity markers⁹ | X | X | X | X | X | X | X | X | X | X | X |
| PK samples¹⁰ | | X | X | X | X | X | X | X | X | X | X |
| ADA samples¹¹ | | X | X | X | X | X | X | X | X | X | X |
| B cell gene signature¹¹ | X | X | | | | | | X | X | X | X |
| PD samples^{11,13} | X | X | X | X | X | X | X | X | X | X | X |
| Receptor Occupancy¹² | | X | X | X | X | X | X | X | X | X | X |
| 12-lead ECG (local) | X | X | X | X | X | X | X | X | X | | X |
| hSLEDAI, ssPGA, SFI, mSFI, BILAG, | X | X | X | X | X | X | X | X | X | X | X |
| CLASI, CGIC | | | | | | | | | | | |
| 28 tender and swollen joint counts | X | X | X | X | X | X | X | X | X | X | X |
| SF-36, FACIT-Fatigue, PGIC/PGIS | X | X | X | X | X | X | X | X | X | X | X |
| Administer methylprednisolone acetate | X | | | | | | | | | | |
| (Depo-Medrol ® or equivalent)¹⁴ | | | | | | | | | | | |
| Study drug administration¹⁵ | | X | X | X | X | X | X | | | | |

Abbreviations: ADA = anti-drug antibodies; ANA = antinuclear antibody; anti-dsDNA = anti-double-stranded deoxyribonucleic acid; BILAG = British Isles Lupus Assessment Group; CGIC = Clinician's Global Impression of Change; CLASI = Cutaneous Lupus Erythematous Disease Area and Severity Index; COVID = Corona virus disease 2019; ECG = electrocardiogram; ENA = extractable nuclear antigen antibody; EOS = end of study; ET = early termination; FACIT = Functional Assessment of Chronic Illness Therapy; FSH = follicle stimulating hormone; GPI = glycoprotein I, HBcAb = hepatitis B core antibody, HbsAb = hepatitis B surface antibody, HbsAg = hepatitis B surface antigen, HBV = hepatitis B virus; HCV = hepatitis C virus; HIV = human immunodeficiency virus; LH = luteinizing hormone; mSFI = modified SFI; mRNA = messenger ribonucleic acid; PD = pharmacodynamics; PGIC = Patient Global Impression of Change in Clinical Status; PGIS = Patient Global Impression of Change in Disease Severity; PK = pharmacokinetics; PRO = patient-reported outcome; RNA = ribonucleic acid; SFI = SELENA-SLEDAI Flare Index; SF-36 = Short Form 36; SLE = systemic lupus erythematosus; hSLEDAI = hybrid Systemic Lupus Erythematosus Disease Activity Index; ssPGA = SELENA-SLEDAI Physician's Global Assessment; Sm = Smith; RNP = ribonucleoproteins; SSA = Sjögren Syndrome A; SSB = Sjögren Syndrome B; TB = tuberculosis; TBNK = T cell, B cell, natural killer (NK) cell; WOCBP = women of child-bearing potential.
Footnotes:
¹Flare Assessment visits are conducted when patients experience symptoms that require assessments for a potential flare.
²Study personnel will record all concomitant medications taken by patients, including prescription and nonprescription medications and any dietary supplements (including vitamins and minerals), nutraceuticals, herbal medicines, traditional Chinese medicines, ayurvedic remedies, and any other products.
³Vital signs include temperature, heart rate, respiratory rate, and blood pressure. On dosing days, vital signs should be performed before the study druginfusion and approximately 30 minutes after the end of infusion.
⁴A complete physical exam includes (at a minimum) assessments of the head, eyes, ears, nose, throat, skin, cardiovascular, mucocutaneous, respiratory, musculoskeletal, lymphatic, gastrointestinal, and neurological systems.
⁵A brief physical examination must be complete enough to be suitable for SLE instrument scoring. At a minimum, assessments will include mucocutaneous, respiratory, cardiovascular, gastrointestinal, and musculoskeletal.
⁶A list of all clinical hematology, serology, coagulation, and urinalysis parameters.
⁷Including HbsAg, HbsAb, HbcAb (total IgM and IgG; if positive, need HBV DNA performed), HCV RNA, HIV 1&2 (and HIV confirmation, if applicable), and QuantiFERON TB test (QuantiFERON may be repeated locally if indeterminant).
⁸SLE serology tests include ANA, ENA (including antibodies against SSA, SSB, Sm, RNP), anti-cardiolipin antibodies (IgA, IgG, and IgM), anti-beta 2 GPIantibodies (IgA, IgG, and IgM), and the lupus anticoagulant.
⁹SLE Disease Activity Markers: serum anti-dsDNA antibodies, complement components C3 and C4, and total serum immunoglobulin profile.
¹⁰At all sites, blood samples for PK analysis will be collected within 30 minutes pre-dose and within 5 minutes after the end of infusion. The start of intravenous infusion is designated as time 0. The duration of infusion is 2 hours. For patients at all Hong Kong sites (and other select patients), additional samples will be collected at the Day 1 and Day 141 visits, at the following timepoints: approximately 6, 24, 36, 48, 72, 120, 168, and 336 hours post-dose.
¹¹Blood samples for RNA and B cell gene signature, PD analysis, and ADA will be collected prior to infusion of study treatment.
¹²Receptor occupancy (if performed) will be conducted in select patients in the US. If performed, blood samples for receptor occupancy analysis will be collected within 30 minutes pre-dose. Additional samples will be collected at the Day 1 and Day 141 visits, at the following timepoints: approximately 6, 24, 72, 168, and 336 hours post-dose.
¹³PD samples will include peripheral blood mononuclear cells for immunophenotype (flow), mRNA analysis including CD32B polymorphisms, andexploratory serum markers.
¹⁴At the end of the Screening visit, if no exclusion criteria are known, a patient will receive one or more intramuscular injections of methylprednisoloneacetate (Depo-Medrol ® or equivalent) at a dose of ≥40 mg (maximum total dose 320 mg, maximum 4 injections). The dose will be determined by the Investigator to achieve a clinically significant improvement in SLE signs and symptoms.
¹⁵Study drug will be administered by intravenous infusion over 2 hours. Patients will be observed for at least 2 hours after first infusion and at least 30 minutesafter other infusions.

### Modifications

Modifications and variations of the described methods and compositions of the present disclosure can be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure are intended and understood by those skilled in the relevant field in which this disclosure resides to be within the scope of the disclosure as represented by the following claims.

### INCORPORATION BY REFERENCE

All patents and publications mentioned in this specification are herein incorporated by reference to the same extent as if each independent patent and publication was specifically and individually indicated to be incorporated by reference.

1. A method of treating B cell driven autoimmune and/or allergic diseases, comprising administering to a subject in need thereof an effective amount of B cell inhibitor that is non-depletional, wherein the subject has reduced CD32B signaling compared to a healthy subject, and wherein the B cell inhibitor is capable of agonizing CD32B despite the reduced CD32B signaling.

2. The method of claim 1, wherein the B cell inhibitor is a CD32B×CD79B bi-specific antibody capable of immunospecifically binding an epitope of CD32B and an epitope of CD79B.

3. The method of claim 2, wherein the CD32B×CD79B bi-specific antibody comprises:
(A) a VL_{CD32B} domain that comprises the amino acid sequence of SEQ ID NO: 1;
(B) a VH_{CD32B} domain that comprises the amino acid sequence of SEQ ID NO: 2;
(C) a VL_{CD79B} domain that comprises the amino acid sequence of SEQ ID NO: 3; and
(D) a VH_{CD79B} domain that comprises the amino acid sequence of SEQ ID NO: 4.

4. The method of claim 3, wherein said CD32B×CD79B bi-specific antibody is an Fc diabody comprising:
(A) a first polypeptide chain that comprises the amino acid sequence of SEQ ID NO: 5;
(B) a second polypeptide chain that comprises the amino acid sequence of SEQ ID NO: 6; and
(C) a third polypeptide chain that comprises the amino acid sequence of SEQ ID NO: 7.

5. The method of claim 4, comprising administering the Fc diabody at a dose of between about 5 mg/kg and about 40 mg/kg, and at a dosage regimen of between one dose per 2 to 8 weeks, for a total of 2 to 20 doses.

6. The method of claim 4, comprising administering the Fc diabody at a dose of about 5 to 20 mg/kg, and at a dosage regimen of one dose every 2 to 6 weeks for a total of 5 to 10 doses.

7. The method of claim 4, comprising administering the Fc diabody at a dose of about 10 mg/kg and at a dosage regimen of one dose per 2 to 4 weeks for a total of 6 to 8 doses.

8. The method of claim 4, comprising administering the Fc diabody at a dose of about 10 mg/kg and at a dosage regimen of one dose every 4 weeks for a total of 6 doses.

9. The method of claim 4, wherein the Fc diabody is administered via an intravenous infusion.

10. The method of claim 9, wherein the Fc diabody is administered over a period of about 1-10 hours, or about 2-4 hours, or about 2 hours.

11. The method of claim 8, wherein the Fc diabody is administered via an intravenous infusion.

12. The method of claim 11, wherein the Fc diabody is administered over a period of about 1-10 hours, or about 2-4 hours, or about 2 hours.

13. The method of claim 1, wherein the disease is selected from Systemic Lupus Erythematosus (SLE), Multiple Sclerosis (MS), Rheumatoid Arthritis (RA), Psoriasis, Dermatomyositis/Polymyositis, Sjogren's Syndrome (SS), Primary Vasculitis (e.g. Polymyalgia rheumatica/Giant cell arteritis/Behçets), Graft vs. Host Disease (GVHD), Myasthenia Gravis, Pemphigus, Neuromyelitis Optica, Anti-NMDA receptor encephalitis, Guillain-Barré syndrome, Chronic inflammatory demyelinating polyneuropathy (CIDP), Grave's opthalmopathy, IgG4 Related Disease (IgG4-RD), Idiopathic thrombocytopenic purpura (ITP), Inflammatory Bowel Disease (IBD), and Crohn's Disease.

14. The method of claim 1, wherein the disease is Systemic Lupus Erythematosus.

15. The method of claim 1, wherein the disease is chronic Systemic Lupus Erythematosus.


SEQUENCE LISTING (7 sequences)

SEQ ID NO: 1 - protein, 107 aa, Unknown
  Synthetic
DIQMTQSPSSLSASVGDRVTITCRASQEISGYLSWLQQKPGKAPRRLIYAASTLDSGVPSRFSGSESGTEFTLTISSLQPEDFATYYCLQYFSYPLTFGGGTKVEIK

SEQ ID NO: 2 - protein, 116 aa, Unknown
  Synthetic
EVQLVESGGGLVQPGGSLRLSCAASGFTFSDAWMDWVRQAPGKGLEWVAEIRNKAKNHATYYAESVIGRFTISRDDAKNSLYLQMNSLRAEDTAVYYCGALGLDYWGQGTLVTVSS

SEQ ID NO: 3 - protein, 112 aa, Unknown
  Synthetic
DVVMTQSPLSLPVTLGQPASISCKSSQSLLDSDGKTYLNWFQQRPGQSPNRLIYLVSKLDSGVPDRFSGSGSGTDFTLKISRVEAEDVGVYYCWQGTHFPLTFGGGTKLEIK

SEQ ID NO: 4 - protein, 113 aa, Unknown
  Synthetic
QVQLVQSGAEVKKPGASVKVSCKASGYTFTSYWMNWVRQAPGQGLEWIGMIDPSDSETHYNQKFKDRVTMTTDTSTSTAYMELRSLRSDDTAVYYCARAMGYWGQGTTVTVSS

SEQ ID NO: 5 - protein, 502 aa, Unknown
  Synthetic
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKAPSSSPMEDIQMTQSPSSLSASVGDRVTITCRASQEISGYLSWLQQKPGKAPRRLIYAASTLDSGVPSRFSGSESGTEFTLTISSLQPEDFATYYCLQYFSYPLTFGGGTKVEIKGGGSGGGGQVQLVQSGAEVKKPGASVKVSCKASGYTFTSYWMNWVRQAPGQGLEWIGMIDPSDSETHYNQKFKDRVTMTTDTSTSTAYMELRSLRSDDTAVYYCARAMGYWGQGTTVTVSSGGCGGGEVAALEKEVAALEKEVAALEKEVAALEKGGGNS

SEQ ID NO: 6 - protein, 270 aa, Unknown
  Synthetic
DVVMTQSPLSLPVTLGQPASISCKSSQSLLDSDGKTYLNWFQQRPGQSPNRLIYLVSKLDSGVPDRFSGSGSGTDFTLKISRVEAEDVGVYYCWQGTHFPLTFGGGTKLEIKGGGSGGGGEVQLVESGGGLVQPGGSLRLSCAASGFTFSDAWMDWVRQAPGKGLEWVAEIRNKAKNHATYYAESVIGRFTISRDDAKNSLYLQMNSLRAEDTAVYYCGALGLDYWGQGTLVTVSSGGCGGGKVAALKEKVAALKEKVAALKEKVAALKE

SEQ ID NO: 7 - protein, 227 aa, Unknown
  Synthetic
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK